(19) United States
(12) Patent Application Publication
(10) Pub. No.: US 2026/0355381 A1
(43) Pub. Date: Nov. 12, 2026

(54) METHOD OF WOUND TREATMENT

(71) Applicant: T.J.Smith and Nephew,Limited, Hull (GB)

(72) Inventors: Anthony Dagger, York (GB); Nicholas Fry, York (GB)

(21) Appl. No.: 19/650,180

(22) Filed: Apr. 16, 2026

Related U.S. Application Data
(62) Division of application No. 17/294,979, filed on Nov. 19, 2019, now abandoned.

(30) Foreign Application Priority Data
Nov. 19, 2018 (GB) 1818811.0

(51) Int. Cl. A61L 15/60; A61F 13/00; A61F 13/05; A61L 15/28; A61L 15/42; A61L 15/44; A61P 17/02; C07K 14/805; C07K 17/12

(52) U.S. Cl. CPC A61L 15/60; A61F 13/00063; A61F 13/05; A61L 15/28; A61L 15/425; A61L 15/44; A61P 17/02; C07K 14/805; C07K 17/12; A61L 2300/252

(57) ABSTRACT

The disclosed technology relates to a method of treating a wound with a substrate. The disclosed technology further relates to the wound treating substrate including an immobilised protein, the use of the substrate in a wound dressing, and the use of the wound dressing in a method of treating a wound.

### CROSS-REFERENCE TO RELATED APPLICATIONS

This application is a divisional of U.S. application Ser. No. 17/294,979, filed May 18, 2021, which is a U.S. national stage application of International Patent Application No. PCT/EP2019/081765, filed Nov. 19, 2019, which claims priority to U.K. Provisional Application No. 1818811.0, filed on Nov. 19, 2018; the disclosure of which is hereby incorporated by reference in its entirety.

### FIELD

The disclosed technology relates to a method of immobilising a protein on a substrate. The disclosed technology further relates to the substrate comprising the immobilised protein, the use of the substrate in a wound dressing, and the use of the wound dressing in a method of treating a wound.

### BACKGROUND

Wound healing is known to be enhanced by the delivery of oxygen to a wound. Delivery of oxygen may be achieved by a number of different techniques. For example, oxygen delivery may be accomplished by intravenous injection of oxygen binders such as haemoglobin into the blood stream, or by spray (EP2550973, published 30 Jan. 2013, Sander et al.). Oxygen may be delivered by incorporating into a dressing a haemoglobin as an oxygen binder as exemplified in US2016/175478, published 23 Jun. 2016, Zal et al.). Other device orientated oxygen delivery techniques include hyperbaric oxygen therapy (HBOT) chambers of varying sizes, or topical oxygen delivery systems such as an oxygen concentrator, or topical oxygen therapy (TOT). Device orientated techniques such as HBOT, TOT, or oxygen concentrators are large pieces of equipment that are permanently located at one location, or are cumbersome for patients to transport during use.

Proteins such as haemoglobin are complex because the protein needs to be encapsulated to ensure a patient's immune system does not adversely react with the protein, or its cellular constituents.

In addition, proteins such as haemoglobin are sensitive to environmental and biological conditions. Modifying the conditions outside of living organism ‘living’ conditions may then result in the protein denaturing or otherwise becoming non-usable.

In contrast, substrates used in wound dressings often require processing conditions out with ‘living conditions’. Thus if a protein is to be incorporated and/or placed on a wound dressing layer the substrate would need to be processed within a relatively narrow range of processing conditions.

### SUMMARY

Embodiments of the present disclosure are directed to a method of immobilising a protein on a substrate and a substrate comprising an immobilised protein. Embodiments of the present disclosure are also directed to dressings comprising the substrate comprising an immobilised protein as well as methods of treatment for wounds using both the substrates comprising an immobilised protein and dressings according to the invention.

According to a first embodiment of the invention there is provided a method of immobilising a protein on a substrate wherein the method comprises the steps of (i) dispersing the protein in an aqueous medium to form a first dispersion, (ii) introducing a water miscible non-aqueous solvent to the first dispersion to form a second dispersion; (iii) contacting the second dispersion with a substrate to form an intermediate substrate; and (iv) freeze drying and/or lyophilising the intermediate substrate to form a substrate having the protein immobilised thereon.

Preferably the temperature in step (ii) is maintained below 40 °C.

Preferably the ratio of the water miscible non-aqueous solvent to water in the second dispersion is less than 50:50 volume:volume, preferably less than 45:55 volume:volume, preferably less than 40:60 volume:volume.

Preferably the substrate is a gelling substrate.

Preferably the protein comprises haemoglobin or a derivative thereof.

According to a second embodiment of the invention there is provided a substrate having a protein immobilised thereon produced according to the method of the first embodiment.

According to a third embodiment of the invention there is provided a physiologically acceptable gelling substrate comprising haemoglobin or a derivative thereof wherein the haemoglobin or derivative thereof is immobilised and stable on the substrate, and wherein the substrate is in a pre-gelled state.

According to a fourth embodiment of the invention there is provided a wound dressing comprising as the wound contacting component a substrate according to the third embodiment of the invention or a substrate having a protein immobilised thereon produced according to the method of the first embodiment of the invention.

According to a fifth embodiment of the invention there is provided a method of treating a wound comprising placing a substrate according to the second embodiment of the invention or a substrate having a protein immobilised thereon produced according to the method of the first embodiment of the invention or a wound dressing according to the fourth embodiment of the invention over a wound.

### BRIEF DESCRIPTION OF DRAWINGS

Embodiments of the present disclosure will now be described hereinafter, by way of example only, with reference to the accompanying drawings in which:

FIG. 1 illustrates a view through an embodiment of a wound dressing according to the invention;

FIG. 2A illustrates a plan view of the dressing of FIG. 1;

FIG. 2B illustrates a perspective view of the dressing of FIG. 1

FIG. 3A illustrates an embodiment of a wound dressing;

FIG. 3B illustrates another embodiment of a wound dressing; and

FIG. 4 illustrates a top view of an embodiment of a wound dressing.

### DESCRIPTION OF EMBODIMENTS

The method of immobilising a protein on a substrate according to the invention comprises the steps of (i) dispersing the protein in an aqueous medium to form a first dispersion, (ii) introducing a water miscible non-aqueous solvent to the first dispersion to form a second dispersion; (iii) contacting the second dispersion with a substrate to form an intermediate substrate; and (iv) freeze drying and/or lyophilising the intermediate substrate to form a substrate having the protein immobilised thereon.

By dispersing the protein first in an aqueous medium and subsequently adding a water-miscible non-aqueous solvent the protein remains stable. The substrate may be subsequently contacted with the dispersion and then dried whilst maintaining the nature of the substrate. Preferably the amount of water-miscible solvent added is such that the ratio of water to water-miscible solvent is less than 50:50 by volume, preferably less than 40:60 by volume.

Without wishing to be bound by theory, it is believed that by using a mixture of an aqueous medium and a water-miscible non-aqueous solvent, the substrate is not fully wetted and as a result may be dried and returned to its pre-wetted state. The term “not fully wetted” should be understood to mean that the substrate is not sufficiently wetted or swollen to lose its form so that on drying the shape of the fibres or other constituent parts is retained thus avoiding coalescence of the fibres or other constituent parts and formation of a rough, i.e. less soft, to touch and less drapable material.

The terms “immobilised” or “immobilising on a substrate” should be understood to mean that, in normal use, the protein remains in the substrate and is not substantially released from the substrate in use. For example, less than 10% of the protein immobilised on the substrate may be released from the substrate in use, preferably less than 5%, more preferably less than 2% or less than 1%. The term immobilised is not intended to exclude free rotation of the protein within the substrate.

The substrate having a protein immobilised thereon preferably has a pre-gelled or pre-wetted state. By pre-gelled or pre-wetted state should be understood to mean that the physical properties of the substrate are substantially unchanged by the method of the invention. Often when a substrate is contacted with water and subsequently dried, the typically desirable conformable and soft nature of the substrate is lost and the substrate becomes less conformable and is often brittle. The method of the present invention is advantageous in that the substrate typically retains or regains its pre-wetted or pre-gelled state after the protein has been immobilised on the substrate. For example, gelling substrates can have a protein immobilised thereon and remain soft, fluffy and conformable prior to use. In the case of a wound dressing, the substrate can therefore be applied to a wound directly without requiring pre-treatment such as wetting of the substrate in order to be made conformable.

Where the substrate is a fibrous substrate, by a pre-gelled or pre-wetted state should be understood to mean that the material after processing is a soft fibrous material and that the fibres remain non-coalesced fibres.

Thus, according to a second embodiment of the invention there is provided a substrate having a protein immobilised thereon produced according to the method of the first embodiment.

The method disclosed herein includes in step (ii) introducing a water-miscible solvent to the aqueous medium.

The water-miscible solvent may be any suitable solvent or mixtures thereof, for example, a water-miscible alcohol, acetone, acetonitrile, dimethyl sulfoxide, dimethyl sulfone, acetic acid or a mixture thereof. Preferably the water-miscible solvent is an alcohol, preferably a short chain alcohol. The short chain alcohol may be chosen from a branched or straight-chained C₁-C₄ alcohol, or a branched or straight-chained C₂-C₃ alcohol. The alcohol may be t-butanol, isopropanol or ethanol or a mixture thereof. In one particular embodiment the alcohol is t-butanol or ethanol.

As disclosed herein the water-miscible non-aqueous solvent to water ratio may vary from 70:30 to 30:70, or 60:40 to 40:60, or 55:45 to 45:55, or 50:50 mixture by volume. As will be appreciated by the person skilled in the art, the most suitable water-miscible non-aqueous solvent to water ratio will depend on the nature of the substrate, the water-miscible non-aqueous solvent and the protein. However, preferably the water-miscible non-aqueous solvent to water ratio is less than 50:50 by volume, preferably less than 45:55 by volume or less than 40:60 by volume.

Preferably during step (ii) the temperature is maintained below 40 °C, preferably below 37 °C, below 30 °C, below 20 °C, below 15 °C, below 10 °C, below 0 °C, below −10 °C, below −20 °C or below −30 °C. Maintaining a low temperature is important to preserve the stability of the protein. The temperature is preferably maintained above the freezing point of the mixture. It will be appreciated that the freezing point of the mixture will vary depending on the water-miscible non-aqueous solvent selected and the ratio of water-miscible non-aqueous solvent to water. For example, an ethanol:water ratio of 70:30 has a freezing point of ~−54 °C, whereas an ethanol:water ratio of 40:60 has a freezing point of ~−30 °C.

If the introducing of the water-miscible solvent to the aqueous medium is an exothermic process as is the case when the water-miscible solvent is an alcohol, step (ii) may require that the first dispersion is actively chilled during step (ii). Preferably to less than 15 °C, preferably to less than 10 °C, preferably to less than 0 °C.

Preferably the water-miscible non-aqueous solvent is chilled to less than 10 °C prior to contacting the water-miscible non-aqueous solvent with the first dispersion, preferably to less than 0 °C, less than −10 °C or less than −15 °C. Preferably the first dispersion is chilled to less than 15 °C prior to step (ii), preferably to less than 10 °C or less than 0 °C.

Preferably the aqueous medium is at a temperature in the range of 1 °C to 10 °C, or 2 °C to 8 °C prior to contact with the protein.

The aqueous medium may be water. The water may be tap, deionised, demineralised, purified, or sea water.

The aqueous medium may further comprise buffers and/or other additives. The use of buffers and/or other additives can facilitate the processing of the protein and/or substrate for example by stabilising the protein. The most suitable buffer will vary depending on the protein however, suitable buffers include ammonium sulfate, guanidine hydrochloride, urea, HEPES (2-[4-(2-hydroxyethyl)piperazin-1-yl]ethanesulfonic acid), MES (2-(N-morpholino)ethanesulfonic acid) monohydrate, CAPS (N-cyclohexyl-3-aminopropanesulfonic acid), CHES (N-Cyclohexyl-2-aminoethanesulfonic acid), HEPPS (3-[4-(2-hydroxyethyl)piperazin-1-yl]propane-1-sulfonic acid), HEPBS ((N-(2-Hydroxyethyl)piperazine-N′-(4-butanesulfonic acid)), MOPS (3-morpholinopropane-1-sulfonic acid), TRIS (tris(hydroxymethyl)aminomethane) and derivatives thereof.

Suitable additives include salts and stabilisers. Suitable salts include magnesium chloride, sodium chloride, sodium gluconate, sodium acetate, potassium chloride, and calcium chloride. Stabilisers may be used to maintain the quaternary structure and therefore the functionality of the protein, even after lyophilisation. The term “stabiliser” is intended to mean a disaccharide, a polyol and/or an antioxidant. Suitable disaccharides include sucrose, trehalose and raffinose, preferably trehalose. Preferably, the polyols are chosen from mannitol and sorbitol. Preferably, the antioxidant is ascorbic acid.

Maintaining a low temperature during the method of the invention ensures that the protein is not partially or wholly denatured when initially contacted. As will be appreciated by the person skilled in the art, the stability of the protein is dependent on both the temperature of the dispersion medium and also the length of time for which the protein is exposed to the dispersion medium. Maintaining a lower temperature therefore increases the stability of the protein over the timescale of the method of the first aspect of the invention. For example, in the case of haemoglobin, if the temperature is significantly above 15 °C the protein may be susceptible to partial denaturing or wholly denatured over the usual timescales of the method of the first aspect of the invention.

### Protein

The protein may be any protein which it is advantageous to immobilise on a substrate. Preferably the protein may have medical applications.

Typically the protein may be an extracellular material. The protein may be an oxygen binding protein, for example a haemoglobin, a myoglobin, a haemocyanin, a haemerythrin, a chlorocruorin, a vanabin, an erythrocruorin, a pinnaglobin, a leghaemoglobin or a coboglobin. Preferably the oxygen binding protein is chosen from haemoglobin, myoglobin, of a human or animal origin, or modified derivatives thereof.

In one particular embodiment the protein is haemoglobin or a modified derivative thereof.

The haemoglobin may be a chemically modified haemoglobin. The haemoglobin may be modified with crosslinkers such that several haemoglobin molecules may be linked together. The crosslinker may be any suitable crosslinker known to the person skilled in the art. For example the crosslinker may be a polyalkylene glycol or a dialdehyde. The crosslinking may be intermolecular or intramolecular.

In some embodiments the protein may be an extracellular haemoglobin from an invertebrate animal, chosen from the phylum Annelida. The extracellular haemoglobin may be derived from marine worms such as Arenicola marina.

Such a haemoglobin may be advantageous because the haemoglobin comprises multiple oxygen binding sites and is stable as an extracellular protein.

The product disclosed herein typically does not contain plasma and/or cell wall constituents.

When the protein is haemoglobin, the substrate may be used to treat ischemic wounds because, provided the haemoglobin is in fluid communication with the wound site, the haemoglobin is capable of transferring oxygen to the wound site.

Typically the protein has a molecular weight of greater than 30,000 Da, preferably greater than 50,000 Da, greater than 80,000 Da or greater than 100,000 Da.

When the protein is a haemoglobin or a derivative thereof, the protein advantageously has a high molecular weight, for example, greater than 100,000 Da, greater than 500,000 Da, greater than 800,000 Da or greater than 1,000,000 Da. Higher molecular weight haemoglobins or derivatives thereof are typically more stable than their lower molecular weight counterparts.

Suitable proteins include VEG F (vascular endothelial growth factor), PDGF (platelet-derived growth factor), TGF Beta (Transforming growth factor beta), invertebrate haemoglobin, such as M101 (a freeze dried annelid haemoglobin derived from Arenicola marina produced by Hemarina), crosslinked vertebrate haemoglobins, such as Granulox produced by Mölnlycke, and proteases, such as thermolysin.

Preferably the protein is present on the substrate at a dose of at least 0.1 mg/cm², preferably at least 0.5 mg/cm², at least 1 mg/cm², at least 5 mg/cm², or at least 10 mg/cm².

Preferably the protein comprises at least 0.1% by weight of the total dry weight of the protein and substrate, preferably at least 1%, preferably at least 5%, preferably at least 10%, preferably at least 20%, preferably at least 30% or preferably at least 40% by weight.

Preferably the protein comprises less than 60% by weight of the total dry weight of the protein and substrate, preferably less than 50%, preferably less than 40%, preferably less than 30%, preferably less than 20%, preferably less than 10% or preferably less than 5% by weight.

### Substrate

The substrate may be any suitable substrate. Typically the substrate is a physiologically acceptable substrate. Typically the substrate is an absorbent substrate, preferably a gelling and/or superabsorbent substrate.

Without wishing to be bound by theory, it is believed that when an absorbent substrate is contacted with a mixture of an aqueous medium and a water-miscible non-aqueous solvent the water-miscible non-aqueous solvent prevents the absorbent substrate from becoming fully wetted. It is thereby possible to dry the substrate and retain the original pre-wetted physical properties of the substrate.

By gelling substrate is intended to mean a substrate that is capable of absorbing aqueous fluid, such as wound exudate, and which on absorbing said fluid becomes gel-like, moist and slippery. The gelling substrate may be any suitable gelling substrate known in the art, including pectin, alginate, chitosan, hyaluronic acid, other polysaccharides or gum derivatives, chemically-modified celluloses, e.g. carboxymethyl cellulose (CMC), or combinations thereof.

By superabsorbent material is intended to mean a material that is typically capable of absorbing many times its own mass of water, for example up to 200, 300 or more times its own mass of water. Examples of suitable superabsorbent materials include a polysaccharide or modified polysaccharide, a polyvinylpyrrolidone, a polyvinyl alcohol, a polyvinyl ether, a polyurethane, a polyacrylate, a polyacrylamide, collagen, a cellulose, gelatin, or mixtures thereof. Typically the substrate is a porous substrate, typically a fibrous substrate.

The substrate may comprise a polymer matrix. The polymer matrix may comprise a polysaccharide or modified polysaccharide, a polyvinylpyrrolidone, a polyvinyl alcohol, a polyvinyl ether, a polyurethane, a polyacrylate, a polyacrylamide, collagen, or gelatin or mixtures thereof.

In some embodiments the polymer matrix may comprise a polysaccharide or modified polysaccharide.

In some embodiments the polymer matrix comprises a polyacrylate superabsorbent or a combination or blend of two or more different superabsorbent materials.

In some embodiments the polymer matrix may not comprise an alginate.

In another embodiment the polymer matrix may be a cellulose. The cellulose may include hydrophilically modified cellulose such as methyl cellulose, carboxymethyl cellulose (CMC), carboxymethyl cellulose (CEC), ethyl cellulose, propyl cellulose, hydroxyethyl cellulose, hydroxypropyl cellulose, hydroxypropylmethyl cellulose, carboxyethyl sulfonate cellulose, cellulose alkyl sulfonate, or mixtures thereof.

In different embodiments the cellulose may be carboxymethyl cellulose, or cellulose alkyl sulfonate.

In one particular embodiment the cellulose may be carboxymethyl cellulose.

In one particular embodiment the cellulose may be cellulose alkyl sulfonate. The alkyl moiety of the alkyl sulfonate substituent group may have an alkyl group having 1 to 6 carbon atoms, such as methyl, ethyl, propyl, butyl, pentyl or hexyl. The alkyl moiety may be branched or unbranched, and hence suitable propyl sulfonate substituents may be 1- or 2-methyl-ethylsulfonate. Butyl sulfonate substituents may be 2-ethyl-ethylsulfonate, 2,2-dimethyl-ethylsulfonate, or 1,2-dimethyl-ethylsulfonate. The alkyl sulfonate substituent group may be ethyl sulfonate. The cellulose alkyl sulfonate may be described in WO10061225 or US2016/114074, or 2006/0142560 or U.S. Pat. No. 5,703,225.

The cellulose alkyl sulfonates may have varying degrees of substitution, the chain length of the cellulose backbone structure, and the structure of the alkyl sulfonate substituent. Solubility and absorbency are largely dependent on the degree of substitution: as the degree of substitution is increased, the cellulose alkyl sulfonate becomes increasingly soluble. It follows that, as solubility increases, absorbency increases.

The substrate may be in the form of fibers and they may have an absorbency of at least 8 grams per gram (g/g), or at least 9 g/g, or at least 10 g/g of 0.9% saline solution. The absorbency may be measured by the following method.

The fiber was cut into a 2-3 mm flock, and 0.5 g of cut fiber was placed in a 100 ml screw-top jar. 50 ml of test liquid (e.g., 0.9% saline, typically used to simulate the ionic strength of wound fluid) was added, and the jar shaken for 30 seconds to disperse the flock. The dispersion was then filtered through a 47 mm Buchner funnel fitted with a 42.5 mm diameter Whatman No. 4 filter paper, using a vacuum pump, with vacuum set to be greater than 0.8 bar for one minute. Then the fiber dispersion was removed and weighed. Fiber free absorbency is calculated using the following formula:

$$absorbency ( g / g ) = [ \frac{wet\_dispersion \_weight ( g )}{dry\_flock \_weight ( g )} ] - 1$$

The calculation is also disclosed in [0077] of US2016/0114074).

### Freeze Drying/Lyophilisation

The intermediate substrate is freeze dried or lyophilised to yield the substrate having an immobilised protein thereon. By freeze drying the intermediate substrate, the protein is not subjected to a high temperature as would be the case in conventional dehydration methods and the solid solvent is removed by lowering the pressure and subliming the solvent. As a result the risk of denaturing the protein is eliminated. The resulting substrate is typically substantially free of unbound water. The skilled person will understand that the temperature required to carry out freeze drying or lyophilisation of the intermediate substrate will depend on the freezing point of the water-miscible non-aqueous solvent and water mixture used. Freeze drying would typically be carried out below the freezing point of the mixture. The freeze drying is preferably carried out at a temperature of −5 °C to −100 °C, preferably −50 °C to −90 °C, typically −80 °C.

After the freeze drying process the temperature of the substrate is typically raised to above 0 °C over a period of at least one hour,

### Wound Dressing

Embodiments disclosed herein relate to apparatuses and methods of treating a wound with or without reduced pressure, including for example a source of negative pressure and wound dressing components and apparatuses. The apparatuses and components comprising the wound overlay and packing materials or internal layers, if any, are sometimes collectively referred to herein as dressings. In some embodiments, the wound dressing can be provided to be utilized without reduced pressure.

Some embodiments disclosed herein relate to wound therapy for a human or animal body. Therefore, any reference to a wound herein can refer to a wound on a human or animal body, and any reference to a body herein can refer to a human or animal body.

The disclosed technology may relate to preventing or minimizing damage to physiological tissue or living tissue, or to the treatment of damaged tissue e.g., a wound as described above.

As used herein the expression “wound” may include any injury to living tissue and may be caused by a cut, blow, or other impact, typically one in which the skin is cut or broken. A wound may be a chronic or acute injury. Acute wounds occur as a result of surgery or trauma. They move through the stages of healing within a predicted timeframe. Chronic wounds typically begin as acute wounds. The acute wound becomes a chronic wound when it does not follow the healing stages resulting in a lengthened recovery. It is believed that the transition from acute to chronic wound can be due to a patient being immuno-compromised.

Chronic wounds may include for example: Venous ulcers: Venous ulcers usually occur in the legs, account for the majority of chronic wounds, and mostly affect the elderly, Diabetic ulcers (typically foot or ankle ulcers, Peripheral Arterial Disease, Pressure ulcers, or Epidermolysis Bullosa (EB).

Examples of other wounds include, but are not limited to, abdominal wounds or other large or incisional wounds, either as a result of surgery, trauma, sternotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

The wound may also include a deep tissue injury. The deep tissue injury is a term proposed by the National Pressure Ulcer Advisory Panel (NPUAP) to describe a unique form of pressure ulcers. These ulcers have been described by clinicians for many years with terms such as purple pressure ulcers, ulcers that are likely to deteriorate and bruises on bony prominences.

The wound may also include tissue at risk of becoming a wound as discussed above. For example, tissue at risk may include tissue over a bony protuberance (at risk of deep tissue injury/insult), pre-surgical tissue (e.g. knee) that may has the potential to be cut (for joint replacement/surgical alteration/reconstruction).

In some embodiments the disclosed technology relates to a method of treating a wound with the technology disclosed herein in conjunction with one or more of the following: advanced footwear, turning a patient, offloading examples such as diabetic foot ulcers, treatment of infection, systemix, antimicrobial, antibiotics, surgery, removal of tissue, affect blood flow, physiotherapy, exercise, bathing, nutrition, hydration, nerve stimulation, ultrasound, electrostimulation, oxygen therapy, microwave therapy, active agents ozone, antibiotics, antimicrobials, and the like.

When the protein is haemoglobin, the disclosed technology may be of particular use in conjunction with oxygen therapy. In oxygen therapy, oxygen is concentrated at the wound site to aid healing. Suitable oxygen concentrator devices for use in conjunction with a substrate having a protein immobilised thereon according to the second aspect of the invention include TransCu O₂ from EO₂, San Antonio, Texas, Natrox from Inotec AMD, Cambridge, and Epiflo form Ogenix, Fort Lauderdale, Florida.

The wound may be treated using topical negative pressure and/or traditional advanced wound care i.e., not aided by the using of applied negative pressure (may also be referred to as non-negative pressure therapy). When the protein is haemoglobin, the wound may be treated using oxygen therapy.

Advanced wound care may include use of an absorbent dressing, an occlusive dressing, use of an antimicrobial and/or debriding agents in a wound dressing or adjunct, a pad e.g., a cushioning or compressive therapy (such as stocking or bandages).

In some embodiments treatment of such wounds can be performed using traditional wound care, wherein a dressing can be applied to the wound to facilitate and promote healing of the wound.

In some embodiments the disclosed technology relates to a method of manufacturing a wound dressing comprising providing a wound dressing as disclosed herein.

The wound dressings that may be utilized in conjunction with the disclosed technology include any known dressing in the art. The technology is applicable to negative pressure therapy treatment as well as non-negative pressure therapy treatment. When the protein is haemoglobin, the technology may be particularly applicable to topical oxygen therapy. Without wishing to be bound by theory, it is believed that the immobilised haemoglobin facilitates oxygen transfer by cascading oxygen between the haemoglobin sites across the substrate.

The invention further provides a wound dressing which comprises or consists of the substrate having a protein immobilised thereon produced according to the method of the first aspect of the invention. Thus according to a second aspect of the invention there is provided a wound dressing comprising substrate having a protein immobilised thereon produced according to the method of the first aspect of the invention.

This is particularly advantageous where the protein is a haemoglobin or a derivative thereof because the haemoglobin can be used to provide oxygen to an ischemic or hypoxic wound.

Thus, according to a third aspect of the invention is provided a physiologically acceptable gelling substrate comprising haemoglobin or a derivative thereof wherein the haemoglobin or derivative thereof is immobilised and stable on the substrate, and wherein the substrate is in a pre-gelled state.

By immobilising haemoglobin on a physiologically acceptable gelling substrate, a product is formed which, on contact with fluid, can act to transfer oxygen down a concentration gradient and thereby treat hypoxic or ischemic wounds.

Because the substrate is in a pre-gelled state, the substrate is conformable and may be easily applied to a wound such that the substrate is in intimate contact with the wound site.

Because the substrate is a gelling substrate, the substrate does not irritate the wound or cause maceration of the healthy skin around the wound. The gelling nature of the substrate also means that the dressing may be easily removed from the wound without causing further irritation.

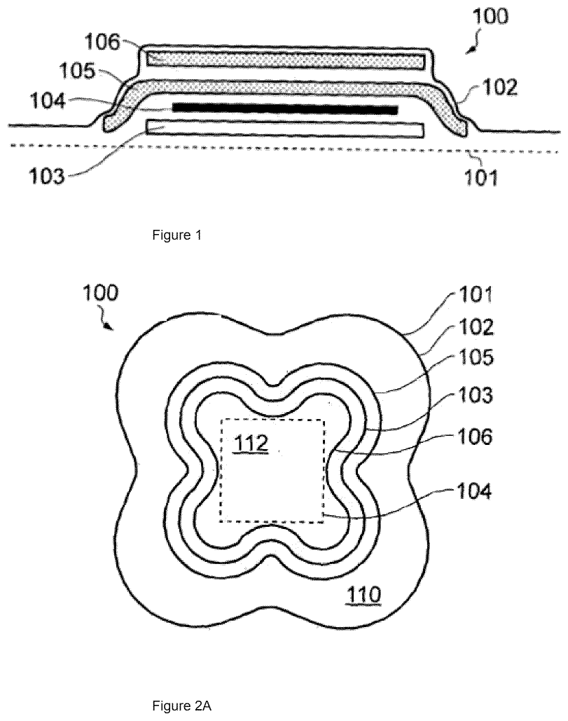

Referring first to FIG. 1, there is shown a wound dressing generally designated 1 comprising a substrate having a protein immobilised thereon produced according to the method of the first aspect of the invention or according to the third aspect of the invention.

Typically a wound dressing according to the invention may comprise a further absorbent layer or layers in addition to the substrate having a protein immobilised thereon produced according to the first aspect of the invention. The additional absorbent layer may be a knitted or woven material, a foam, a superabsorbent or a combination thereof.

For the dressings according to the invention, the substrate may be contained between a wound contact layer and a top film.

The wound contact layer can comprise a perforated wound-side adhesive which can be a silicone adhesive, or a low-tack adhesive to minimise skin trauma on removal. The wound contact layer comprises a support material which can be a mesh, a net or a perforated film. It can also comprise a construction adhesive on the pad side, to ensure its intimate contact with the lowest part of the pad, and therefore efficient uptake of fluid from the wound without pooling.

Where the substrate having a protein immobilised thereon according to a first aspect of the invention is a haemoglobin or derivative thereof, the wound dressing preferably does not comprise a wound contact layer and the substrate having a protein immobilised thereon is preferably in direct contact with the wound.

Where the protein immobilised on the substrate is a haemoglobin or derivative thereof, the substrate may be incorporated in a wound dressing particularly for treating ischemic or hypoxic wounds. When the substrate having haemoglobin immobilised thereon is in fluid communication with a wound site, the haemoglobin is able to transfer oxygen to the wound site provided that the substrate is sufficiently close to the wound site. The distance across which oxygen may be transferred is limited to approximately 40 microns. It is therefore preferable for the substrate having haemoglobin immobilised thereon to be the wound contacting layer in a wound dressing and preferably in intimate contact with the wound site.

The top film may be a liquid-impermeable, moisture-vapour permeable, breathable film, which allows moisture to evaporate from the dressing. Where the protein immobilised on the substrate is a haemoglobin or derivative thereof and the substrate may be incorporated in a wound dressing particularly for treating ischemic or hypoxic wounds, the top film is preferably oxygen permeable so that oxygen may be continuously supplied to the wound site down an oxygen concentration gradient.

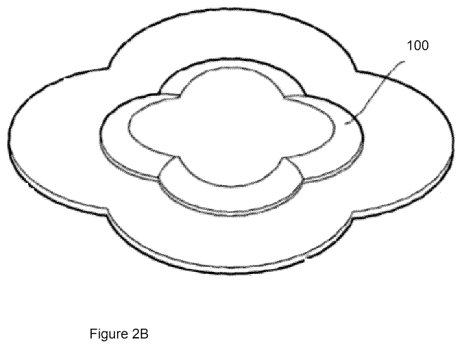

FIGS. 1, 2A and 2B respectively show a schematic cross-sectional view, a plan view and a perspective view of a wound dressing according to an embodiment of the present disclosure. The wound dressing 100 includes a number of layers that are built up in a generally laminar fashion to form a dressing having a relatively planar form. The wound dressing 100 includes a border region 110 extending around the outer periphery of the dressing. The central region may be predetermined to suit a particular wound or particular wound type. There may be no border region required. Here the border region has the general function of providing an area for sealingly engaging with a patient's skin surrounding a wound site to form a sealed cavity over the wound site. The central region is the location of further functional elements of the wound dressing.

The dressing 100 includes a top film 102 and the substrate having a protein immobilised thereon 103.

Further components of the wound dressing 100 include:

A perforated wound contact layer 101.

A layer of absorbent material 105, for example a polyurethane hydrocellular foam, of a suitable size to cover the recommended dimension of wounds corresponding to the particular dressing size chosen

A layer of activated charcoal cloth 104 of similar or slightly smaller dimensions than 103, to allow for odour control with limited aesthetic impact on the wound side.

A layer of three-dimensional knitted spacer fabric 106, providing protection from pressure, while allowing partial masking of the top surface of the superabsorber, where coloured exudate would remain. In this embodiment this is of smaller dimension (in plan view) than the layer 105, to allow for visibility of the edge of the absorbent layer, which can be used by clinicians to assess whether the dressing needs to be changed.

The wound contact layer 101 may be a perforated polyurethane film that is coated with a skin-compatible adhesive, such as pressure sensitive acrylic adhesive or silicone adhesive (not shown). Alternatively the wound contact layer may be formed from any suitable polymer, e.g. silicone, ethylvinyl acetate, polyethylene, polypropylene, or polyester, or a combination thereof. The skin-compatible adhesive is coated on the lower side of the layer 101, i.e. the side that is to contact the patient.

As set out above, the wound contact layer may preferably be absent.

The substrate having a protein immobilised thereon 103 extends over the central region 112 of the dressing.

The foam may be any suitable polymer foam. The foam is aptly a highly conformable hydrophilic foam, aptly an open celled foam, and more aptly the foam is a mixture of open and closed cells.

It is desirable that the foam layer absorbs the wound exudate rapidly. Such rapid absorption prevents undesirable pooling of exudate between the dressing and the wound.

The odour-removing layer of activated charcoal cloth 104 is provided over the layer of foam 103. In this embodiment the activated charcoal layer is about the same length and depth as the foam layer and therefore lies over the foam layer to cover about the same area. The layer may be of Zorflex® cloth available from Chemviron Carbon, for example. Alternative suitable materials are manufactured by MAST under the trade name C-TeX®.

The function of the odour-removing layer is to help prevent or reduce odour originating from the wound from transmitting out of the dressing.

The layer of absorbent material 105 is provided over the odour-removing layer 104. The absorbent layer 105 extends fully over the layer 104, as well as over the side portions of both the odour-removing layer 104 and the substrate having a protein immobilised thereon 103.

The layer 105 forms a reservoir for fluid, particularly liquid, removed from the wound site and draws those fluids towards a cover layer 102. The material of the absorbent layer also prevents liquid collected in the wound dressing from flowing freely once in the dressing structure. The second absorbent layer 105 also helps distribute fluid throughout the layer via a wicking action so that fluid is drawn from the wound site and stored throughout the absorbent layer, i.e. transferring and locking in the liquid. This prevents agglomeration in areas of the absorbent layer. The capacity of the absorbent material should be sufficient to manage the exudate flow rate of a wound for the predetermined life of the dressing, whether the wound is acute or chronic. Again, in combination with the substrate having a protein immobilised thereon, the layer 105 aptly should not cause the wound to become completely dry. This might occur if, for example, the superabsorbent material were to dry out the foam layer and then subsequently the wound area.

The shielding layer 106 is a layer having a 3-dimensional structure that may include open cell foam (e.g. Alleyvn™ foam by Smith & Nephew, Biatain foam by Coloplast or Advanced Medical Devices' ActivHeal foam), a knitted or woven spacer fabric (for example Baltex 7970 weft knitted polyester or Baltex XD spacer fabric or Surgical Mesh's Polyester felt or Polyester mesh) or a non-woven fabric (e.g. Fiberweb's S-tex or Securon). Alternatively the shielding layer may be a completely opaque polymer film having cut-out windows or perforations, for example (e.g. SNEF's H514 or H518 blue net).

Another function of the shielding layer 106 may be for pressure distribution and impact protection. For example, if the patient accidentally knocks the wound area, leans on the wound area or another cause applies a pressure to the dressing covering a wound. Aptly the shielding layer is provided closer to where the pressure is being applied than other layers of the dressing.

The shielding layer 106 acts as a pressure spreading component, receiving a pressure on one side thereof (possibly a point force) and spreading the pressure over a wider area, thus reducing the relative pressure received on the other side of the shielding layer. As such, the level of pressure felt by the patient at the wound site is reduced.

The top film 102 is a cover layer for covering the lower layers of the dressing, helping to encapsulate the layers between the wound contact layer and the top film. The top film 102 is in this case a layer of polyurethane, Elastollan (trade name) SP9109 manufactured by BASF. The top film may be coated with any suitable adhesive. Aptly the adhesive will be a pressure sensitive adhesive e.g. acrylic adhesive or silicone adhesive.

As such, the top film 102 helps to ensure that the dressing remains breathable, i.e. allows a proportion of fluid absorbed in the dressing to be evaporated via the outer surface of the dressing and where the protein is haemoglobin or a derivative thereof, allows transport of oxygen into the dressing. In this way certain fluid content of the exudate can be transpired from the dressing, reducing the volume of remaining exudate and increasing the time before the dressing becomes full. Also, the top cover 102 helps to ensure that the border region 110 of the dressing remains breathable, i.e. allows a patient's normal skin perspiration to be evaporated through the dressing, which helps in preventing or minimising skin maceration.

The outer layer of dressings of the present disclosure when present can be a continuous conformable film. The continuous moisture vapour transmitting conformable film outer layer of the wound dressing may be used to regulate the moisture loss from the wound area under the dressing and also to act as a barrier to bacteria so that bacteria on the outside surface of the dressing cannot penetrate to the wound area. Suitable continuous conformable films will have a moisture vapour transmission rate of at least 300, aptly from 300 to 5000 grams preferably 500 to 2000 grams/square meter/24 hrs at 37.5 C at 100% to 10% relative humidity difference. Such moisture vapour transmission rate of the continuous film allows the wound under the dressing to heal under moist conditions without causing the skin surrounding the wound to macerate.

In use, a wound dressing as described above would be applied to a wound site of a patient with the surface of the substrate according to the invention facing the wound site. Any wound exudate, blood or other wound fluid would travel into the dressing via the substrate according to the invention and sequential layers above the substrate according to the invention. Fluid would permeate through the foam layer, the activated charcoal layer, and then reach the absorber layer at which point preferably the liquid would not go any further and be retained by the absorber layer. On the other hand, gas and moisture vapour, and in particular oxygen where the protein is haemoglobin or a derivative thereof, would be able to permeate further via the shielding layer and/or top film.

The wound facing surface of a wound dressing may be provided with a release coated protector (not shown in the figures), for example a silicon-coated paper. The protector covers the wound contacting side of the dressing prior to application to a patient, and can be peeled away at the time of use.

Various modifications to the detailed arrangements as described above are possible. For example, dressings according to the present disclosure do not require each of the specific layers as described above with respect to FIG. 1. Dressings may include only one layer, or any combination of the layers described above. Alternatively or additionally, the materials of the layers described above may be combined into a single layer or sheet of material to perform the functions of each layer by a single layer.

As noted above, each of the layers described may be used to give one or more function to the wound dressing. As such, each of the layer materials may be used separately or in any combination such that each material provides the given function.

The wound contact layer described above is an optional layer. If used, a wound contact layer may be of any suitable material, such as polyethylene (or polyurethane as described above) or other suitable polymer, and may be perforated for example by a hot pin process, laser ablation process, ultrasound process or in some other way so as to be permeable to fluids.

Although the dressing described above has been described having a border region and a central region this need not be the case. The dressing may be provided without an adhesive layer for attachment to the skin of a patient. Rather, another means may be provided for locating the dressing at the correct position over a wound, such as adhesive tape or a tied bandage.

The relative widths of the various layers may be all the same or different to those as shown in the figures.

A wound dressing may be formed by bringing together the required layers. The method may include bringing layers together with adhesive over part or all of a layer. The method may be a lamination process.

Alternatively a wound dressing may be formed by bringing together layers as described with respect to FIG. 1, in a contiguous laminar stack, and adhering the top film to the wound contact layer in a border region.

The methods above may include bringing layers together with adhesive over part or all of a layer. The method may be a lamination process.

Alternatively a wound dressing may be formed by bringing together layers as described with respect to FIG. 1, in a contiguous laminar stack, and adhering the top film to the wound contact layer in a border region.

Any of the dressing embodiments disclosed herein can be used in with a source of negative pressure, such as a pump. Any of the dressing embodiments disclosed herein can also be used with a pump and a fluid or waste collection canister that can be put in fluid communication with the pump and the dressing so that the pump draws fluid or waste from the wound into the collection canister.

Additionally, in any embodiments, the pump can be a piezoelectric pump, a diaphragm pump, a voice coil actuated pump, a constant tension spring actuated pump, a manually actuated or operated pump, a battery powered pump, a DC or AC motor actuated pump, a combination of any of the foregoing, or any other suitable pump.

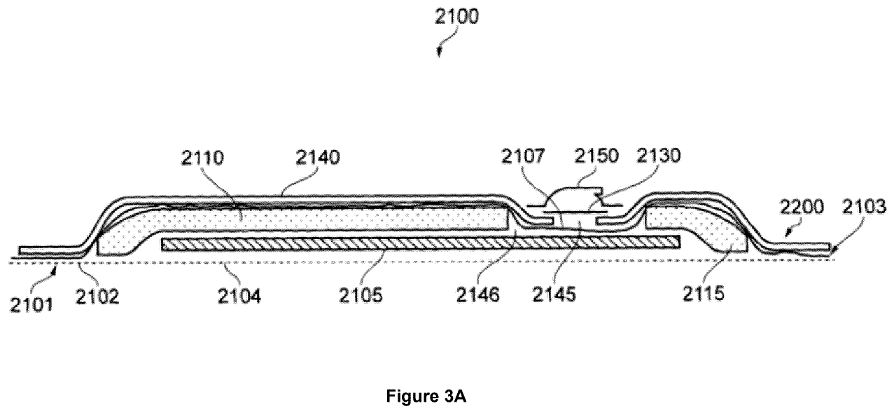

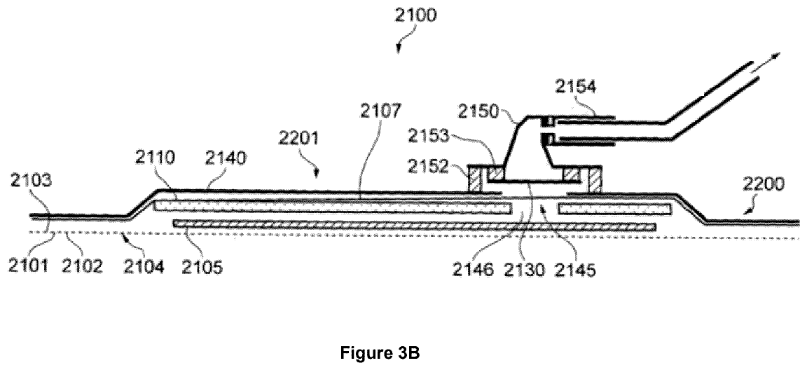

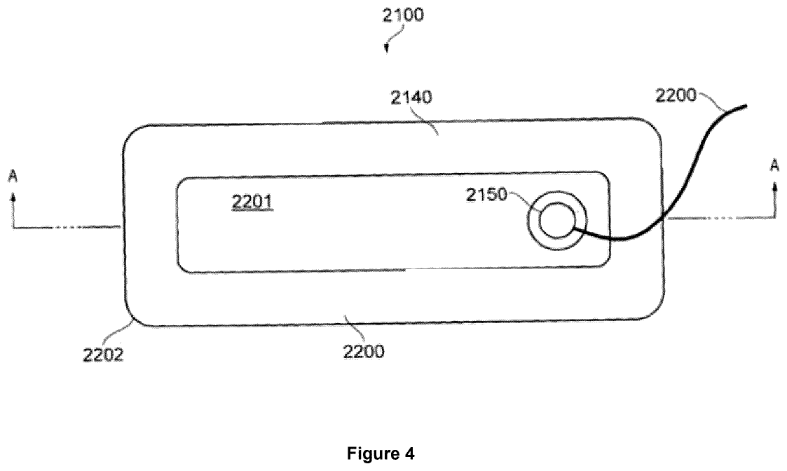

FIGS. 3A-B illustrate cross sections through a wound dressing 2100 according to an embodiment of the disclosure. A plan view from above the wound dressing 2100 is illustrated in FIG. 4 with the line A-A indicating the location of the cross section shown in FIGS. 3A and 3B. It will be understood that FIGS. 3A-B illustrate a generalized schematic view of an apparatus 2100. It will be understood that embodiments of the present disclosure are generally applicable to use in TNP therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

The wound dressing 2100, which can alternatively be any wound dressing embodiment disclosed herein including without limitation wound dressing 100 or have any combination of features of any number of wound dressing embodiments disclosed herein, can be located over a wound site to be treated. The dressing 2100 forms a sealed cavity over the wound site.

When a wound packing material is used, once the wound dressing 2100 is sealed over the wound site, TNP is transmitted from a pump through the wound dressing 2100, through the wound packing material, and to the wound site. This negative pressure draws wound exudate and other fluids or secretions away from the wound site. The wound contact layer 2102 can be a polyurethane layer or polyethylene layer or other flexible layer which is perforated, for example via a hot pin process, laser ablation process, ultrasound process or in some other way or otherwise made permeable to liquid and gas. The wound contact layer has a lower surface 2101 and an upper surface 2103. The perforations 2104 are through holes in the wound contact layer which enables fluid to flow through the layer.

A layer 2105 of the substrate according to the second embodiment of the invention can be located above the wound contact layer. A transmission layer 2110 is provided above the substrate according to the second aspect of the invention 2105. This transmission layer, 2110 allows transmission of fluid including liquid and gas away from a wound site into upper layers of the wound dressing. In particular, the transmission layer 2105 ensures that an open air channel can be maintained to communicate negative pressure over the wound area even when the absorbent component has absorbed substantial amounts of exudates. The layer should remain open under the typical pressures that will be applied during negative pressure wound therapy as described above, so that the whole wound site sees an equalized negative pressure. The layer 2105 is formed of a material having a three dimensional structure. For example, a knitted or woven spacer fabric (for example Baltex 7970 weft knitted polyester) or a non-woven fabric could be used. Other materials could of course be utilized. With reference to FIGS. 3A and 38, a masking or obscuring layer 2107 can be positioned beneath the cover layer 2140. In some embodiments, the masking layer 2107 can have any of the same features, materials, or other details of any of the other embodiments of the masking layers disclosed herein, including but not limited to having any viewing windows or holes. Additionally, the masking layer 2107 can be positioned adjacent to the cover layer, or can be positioned adjacent to any other dressing layer desired. In some embodiments, the masking layer 2107 can be adhered to or integrally formed with the cover layer. In some embodiments the masking layer 2107 may optionally contain a hole (not shown) directly adjacent to the port 2150 to improve air flow through the layer.

A gas impermeable, but moisture vapour permeable, cover layer 2140 can extend across the width of the wound dressing, which can be any wound dressing embodiment disclosed herein including without limitation dressing embodiment 100 or have any combination of features of any number of wound dressing embodiments disclosed herein. The cover layer, which may for example be a polyurethane film (for example, Elastollan SP9109) having a pressure sensitive adhesive on one side, is impermeable to gas and this layer thus operates to cover the wound and to seal a wound cavity over which the wound dressing is placed. In this way an effective chamber is made between the cover layer and a wound site where a negative pressure can be established. The cover layer 2140 is sealed to the wound contact layer 2102 in a border region 2200 around the circumference of the dressing, ensuring that no air is drawn in through the border area, for example via adhesive or welding techniques. The cover layer 140 protects the wound from external bacterial contamination (bacterial barrier) and allows liquid from wound exudates to be transferred through the layer and evaporated from the film outer surface. The cover layer 2140 typically comprises two layers; a polyurethane film and an adhesive pattern spread onto the film. The polyurethane film is moisture vapour permeable and may be manufactured from a material that has an increased water transmission rate when wet.

The filter element 2130 may also include an odour absorbent material, for example activated charcoal, carbon fibre cloth or Vitec Carbotec-RT Q2003073 foam, or the like. For example, an odour absorbent material may form a layer of the filter element 2130 or may be sandwiched between microporous hydrophobic membranes within the filter element.

The filter element 2130 thus enables gas to be exhausted through the orifice 2145. Liquid, particulates and pathogens however are contained in the dressing.

The wound dressing 2100 and its methods of manufacture and use as described herein may also incorporate features, configurations and materials described in the following patents and patent applications that are all incorporated by reference in their entireties herein: U.S. Pat. Nos. 7,524,315, 7,708,724, and 7,909,805; U.S. Patent Application Publication Nos. 2005/0261642, 2007/0167926, 2009/0012483, 2009/0254054, 2010/0160879, 2010/0160880, 2010/0174251, 2010/0274207, 2010/0298793, 2011/0009838, 2011/0028918, 2011/0054421, and 2011/0054423; as well as U.S. application Ser. No. 12/941,390, filed Nov. 8, 2010, Ser. No. 29/389,782, filed Apr. 15, 2011, and Ser. No. 29/389,783, filed Apr. 15, 2011. From these incorporated by reference patents and patent applications, features, configurations, materials and methods of manufacture or use for similar components to those described in the present disclosure may be substituted, added or implemented into embodiments of the present application.

In operation the wound dressing 2100 is sealed over a wound site forming a wound cavity. A pump unit applies a negative pressure at a connection portion 2154 of the port 2150 which is communicated through the orifice 2145 to the transmission layer 2105. Fluid is drawn towards the orifice through the wound dressing from a wound site below the wound contact layer 2102. The fluid moves towards the orifice through the transmission layer 2105. As the fluid is drawn through the transmission layer 2105 wound exudate is absorbed into the absorbent component 2110.

Turning to FIG. 4 which illustrates a wound dressing 2100 in accordance with an embodiment of the present disclosure one can see the upper surface of the cover layer 2140 which extends outwardly away from a centre of the dressing into a border region 2200 surrounding a central raised region 2201 overlying the transmission layer 2105 and the absorbent component 2110. As indicated in FIG. 4 the general shape of the wound dressing is rectangular with rounded corner regions 2202. It will be appreciated that wound dressings according to other embodiments of the present disclosure can be shaped differently such as square, circular or elliptical dressings, or the like.

### EXAMPLES

### Example 1

Disks of Durafiber, a cellulose ethyl sulfonate gelling fibrous substrate produced by Smith & Nephew Medical Limited, Hull, were cut out using a clicker press with a 22 mm diameter cutter tool. Aquacel (a carboxymethyl cellulose dressing produced by Convatec) disks (9) and Durafiber disks (9) were placed into separate 12-well culture plates. M101 (a freeze dried annelid haemoglobin derived from Arenicola marina produced by Hemarina) and Granulox (a crosslinked haemoglobin produced by Mölnlycke) solutions were prepared by placing ultra-pure water (18 MΩ) into 150 ml sterilised pots located in a water bath at 4 °C. The solutions were stirred with an overhead stirrer while adding M101 (at 5 °C) or Granulox followed by ethanol (pre-cooled to −18 °C) according to the following compositions:

| | Haemoglobin | Water | Ethanol | Total |
|---|---|---|---|---|
| Process Control | 0 | 38.50 ml | 31.50 ml | 70 ml |
| M101 | 1.155 ml | 37.35 ml | 31.50 ml | 70 ml |
| Granulox | 1.155 ml | 37.35 ml | 31.50 ml | 70 ml |

The haemoglobin solutions were dosed into the Durafiber/Aquacel residing in 22 mm well plates by dosing 1.148 ml of saline to give 0.249 mg/cm² haemoglobin apart from process control which omitted haemoglobin. The dosed well plates were placed into a −80 °C freezer for >16 hours.

The dosed well plates were transferred from the −80 °C freezer to the freeze dryer. After sealing the freeze dryer the vacuum valve was opened to apply vacuum to the sample chamber. The vacuum and temperatures were monitored for ~23 hours.

| Run | Vac | T(shelf) | T(near sample) | |
|---|---|---|---|---|
| time | (mbar) | (°C) | (°C) | Comments |
| 5 | 0.7 | 2 | 12 | Some disks risen |
| | | | | to top in well |
| | | | | plate |
| 10 | 0.59 | 1 | 11 | |
| 15 | 0.36 | 1 | 11 | |
| 20 | 0.31 | 2 | 11 | Aquacel lid lifting |
| | | | | up slightly |
| 30 | 0.25 | 4 | 12 | Ditto for |
| | | | | Durafiber + Aquacel |
| 1.07 | 0.25 | 7 | 13 | Ditto for |
| | | | | Durafiber + Aquacel |
| 2 hrs 14 | 0.25 | 10 | 14 | |
| 3 hrs 50 | 0.18 | 11 | 16 | No movement. All |
| | | | | disks in well |
| | | | | bottom |
| 5 hrs 19 | 0.15 | 15 | 18 | No movement. All |
| | | | | disks in well |
| | | | | bottom |
| 7 hrs 48 | 0.19 | 17 | 19 | No movement. All |
| | | | | disks in well |
| | | | | bottom |
| 23 hrs 44 | 0.18 | 16 | 18 | No movement. All |
| | | | | disks in well |
| | | | | bottom |

The vacuum was released. The samples were removed and vacuum sealed into foil pouches.

### Results

The treated Durafiber/Aquacel disks were removed from foil packs and compared visually and for softness/loftyness/conformability—see appended images.

Due to their greater thickness and absorbency, the Durafiber disks were a little less conformable compared to Aquacel. There was no difference in conformability between the disks (Aquacel+Durafiber) when treated with either Hemarina M101 or Granulox, when compared to their controls or untreated disks. All disks felt soft to the touch.

Untouched disks were dropped into saline and left for 10 minutes. The saline remained coloured and disk colour remained unchanged indicating that the haemoglobin was still substantially present within the disks.

### Example 2—Effect of Aqueous Tert-Butanol Ratio on Durafiber

A twelve-well culture plate was dosed with different aqueous tert-butanol solutions (0.95 ml/well), containing between 0% and 100% tert-butanol. Twelve 22 mm diameter Durafiber discs were placed each into one the twelve wells containing the dosed solution and pressed firmly into the well bottom with tweezers. The dosed aqueous alcohol solutions were removed by freeze drying. An examination of the disks and wells was made (Table 1 & FIG. 1). Below 40% tert-butanol the Durafiber developed some rigidity and lost some of its softness presumably due to excessive fibre swelling which allowed some fibres to coalesce together on drying. Some white residue was left in the wells at 40 to 50% alcohol concentrations, perhaps due to loose fibres or some complete dissolution of a small quantity of the Durafiber which was then left coating the well bottom edges.

TABLE 1
Observations of Durafiber discs treated to freeze drying
process with various water/tert-butanol ratios

| % v/v | |
|---|---|
| Tert-Butanol in Water | Observation & Feel |
| 100 | Soft feel/No white residue left in well |
| 75 | |
| 70 | |
| 65 | |
| 60 | |
| 55 | Soft feel/White residue around well edges |
| 50 | |
| 45 | |
| 40 | |
| 35 | Soft feel/Some rigidity |
| 30 | Rough feel/Very rigid |
| 0 | Rough feel/Solid |

### Example 3—Treatment of Superabsorber with M101

Superabsorber LiquiBlock 40 k (200-1000 um particle size) from Emerging Technologies Inc was used in place of Durafiber or Aquacel. Superabsorber particles (0.5 g) were placed into 10 ml vials and transferred to a fridge (5 °C) for ~60 mins. Pure water at 5 °C (10.67 ml) was placed into a 30 ml jar followed by M101 at 5 °C (0.33 ml), then slowly adding ethanol at −18 °C (9.00 ml) with stirring. The solution was transferred in 2.5 ml aliquots to the vials containing superabsorber particles and stored in a fridge (5 °C) to allow time for the superabsorber to absorb the fluid. After ~60 mins the vials were transferred to a −80 °C freezer for 16 hours then subjected to vacuum sublimation in a precooled freeze dryer over 24 hours. The treated superabsorber was sealed in the vials used during the treatment.

### Results

The 40 k M101 treated and freeze dried material reformed into its original particle size after freeze drying, forming a course reddish powder. Addition of water at a very slight excess to the swelling capacity (200:1) caused the superabsorber particles to swell as expected. The retention of the red colourisation is indicative of intact haemoglobin rather than the brown colour seen with denatured haemoglobin.

### Example 4—Treatment of Gelling Fabric with Thermolysin

The same process method, described above, was followed except that the treatment solution consisted of pure water at 5 °C (11.00 ml) plus Thermolysin (a metalloproteinase enzyme from Smith & Nephew) (0.066 g) and ethanol at −18 °C (9.00 ml). Aliquots of the prepared solution (1.148 ml) were transferred to culture wells containing cold (5 °C) 22 mm disks of Durafiber to yield a Thermolysin dose of 1.0 mg/cm². Further Durafiber disks were treated using the same solution omitting Thermolysin to act as process controls. The treated disks contained within culture well plates were transferred to a −80 °C freezer for >16 hours then transferred to a precooled freeze dryer (Steric Lyovac G77) and subjected to vacuum sublimation for ~24 hours to remove the aqueous ethanol solution used to carry the Thermolysin. The freeze dried disks were heat sealed in vacuum packed aluminium foil pouches.

Results of Durafiber Dosing/Freeze Drying of Thermolysin (1 mg/cm2) into Durafiber

The Thermolysin treated Durafiber disks were tested for their casein potency relative to the Thermolysin dose used. After extraction with either aqueous buffer solution or ethanol the activity was measured at 56.4% and 35.9% respectively, indicating that the extracts had active Thermolysin. The higher activity value seen for the aqueous buffer solution is likely to be due to the greater swelling of Durafiber in aqueous versus ethanolic conditions enabling more Thermolysin to be removed.

TABLE 1
Casein activity measured for Thermolysin-loaded DURAFIBER
and extracted with either buffer or 20% ethanol for 1 h.

| | | | | Rel. to |
|---|---|---|---|---|
| Sample | TLN Extract | | Extract activity | label |
| Description | mg/mL | PU/mL | PU/ml | % |
| Low TLN, TBS | 0.250 | 1845 | 1426.99 ± 30.87 | 77.34 |
| | | | (2.16 | |
| Low TLN, Ethanol | 0.250 | 1845 | 486.25 ± 4.68 | 26.35 |
| | | | (0.96) | |
| TRS, TBS | 0.250 | 1845 | 1831.86 ± 12.16 | 56.44 |
| | | | (0.66) | |
| TRS, Ethanol | 0.250 | 1845 | 2041.5856.14 | 35.93 |
| | | | (2.75} | |
| DURAFIBER, TBS | 0.000 | 0 | 78.06 ± 9.36 | 99.29 |
| | | | (11.98 | |
| DURAFIBER, Ethanol | 0.000 | 0 | 67.48 ± 24.33 | 110.65 |
| | | | (36.05) | |

Casein activity was tested for Thermolysin-loaded DURAFIBER discs that were extracted in either TBS (Tris buffered saline) or 20% ethanol. Discs were loaded with a low or high dose of TLN (thermolysin) and then extracted in designated excipient for 1 h. Samples were further diluted, if necessary, to achieve 0.25 mg/mL TLN. Samples were further diluted to 0.7 μg/mL for the assay. TRS (Thermolysin reference standard) was prepared in TBS for a 5-point calibration curve. Reactions were initiated by the addition of 1 mL of final TLN solution in Tris (~5 PU (protease unit)/mL) to 5 mL of 1.5% (w/v) casein prepared in Substrate TRIS and incubated at 37 °C prior to reaction, and quenched at 30 min. The samples were filtered via Whatman 44 filter paper, and the absorbance at 275 nm was recorded for all filtrate samples. Reactions were measured in duplicate from the same sample preparation. Sample activity is calculated relative to the calibration standard prepared with the TRS. A275 of each standard was plotted against the activity in PU/mL for each standard. Gel sample activity was then calculated with dilution factor correction and reported in kiloPU/g of gel. Standard deviation is reported after the ‘±’ and relative standard deviation is reported in parentheses.

Rel. to Label=percent activity measured relative to the activity calculated for each extract theoretical 0.25 mg/mL

### CONCLUSIONS

An aqueous ethanol dosing and freeze drying process has been shown to successfully immobilise intact haemoglobin (M101 & Granulox) or Thermolysin onto gelling non-woven fabric for potential use within wound dressings. Careful mixing and temperature control prevented the protein denaturing process that is normally seen in the presence of ethanol.

With the embodiments of the present disclosure, a wound dressing is provided that helps improve patient concordance with instructions for use, helps improve patients' quality of life, and also helps a clinician observe and monitor a patient's wound.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Throughout the description and claims of this specification, the singular encompasses the plural unless the context otherwise requires. In particular, where the indefinite article is used, the specification is to be understood as contemplating plurality as well as singularity, unless the context requires otherwise.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” “essentially” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Features, integers, characteristics, compounds, chemical moieties or groups described in conjunction with a particular aspect, embodiment or example of the present disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

The reader's attention is directed to all papers and documents which are filed concurrently with or previous to this specification in connection with this application and which are open to public inspection with this specification, and the contents of all such papers and documents are incorporated herein by reference.

1. A method of treating a wound, comprising:
placing a physiologically acceptable gelling substrate over a wound, the gelling substrate comprising a protein or a derivative thereof, wherein the protein or derivative thereof is immobilized and stable on the gelling substrate, the gelling substrate in a pre-gelled state.

2. The method of claim 1, wherein the gelling substrate is absorbent.

3. The method of claim 1, wherein the gelling substrate comprises cellulose ethyl sulfonate.

4. The method of claim 1, wherein the protein comprises haemoglobin or a derivate thereof.

5. The method of claim 4, wherein the haemoglobin is an annelid derived haemoglobin.

6. The method of claim 1, wherein the protein or protein derivative is immobilized via a process, the process comprising dispersing the protein or protein derivative in an aqueous medium to form a first dispersion.

7. The method of claim 6, wherein the process of immobilizing the protein or protein derivative comprises introducing a water miscible non-aqueous solvent to the first dispersion to form a second dispersion.

8. The method of claim 7, wherein the process of immobilizing the protein or protein derivative comprises contacting the second dispersion with a precursor substrate to form an intermediate substrate.

9. The method of claim 8, wherein the process of immobilizing the protein or protein derivative comprises freeze drying and/or lyophilizing the intermediate substrate to form the gelling substrate.

10. The method of claim 7, wherein the water miscible non-aqueous solvent comprises alcohol.

11. The method of claim 10, wherein the alcohol is selected from the group consisting of t-butanol, isopropanol, and ethanol.

12. The method of claim 7, wherein the ratio of the water miscible non-aqueous solvent to water in the second dispersion is less than 50:50 volume:volume.

13. The method of claim 1, further comprising placing a cover layer over the gelling substrate.

14. The method of claim 13, further comprising applying negative pressure to the wound through the cover layer.

15. The method of claim 14, wherein negative pressure is applied via a negative pressure source.

16. The method of claim 15, wherein the negative pressure source is a vacuum pump.
